(19) United States
(12) Patent Application Publication
(10) Pub. No.: US 2021/0238273 A1
(43) Pub. Date: Aug. 5, 2021

(54) TREATMENT OF ATOPIC DERMATITIS

(71) Applicant: JANSSEN BIOTECH, INC., Horsham, PA (US)

(72) Inventor: John Simard, Austin, TX (US)

(21) Appl. No.: 17/048,865

(22) PCT Filed: Apr. 24, 2019
(86) PCT No.: PCT/US2019/028877

§ 371 Date: Oct. 19, 2020

Related U.S. Application Data
(60) Provisional application No. 62/662,055, filed on Apr. 24, 2018.
(60) Provisional application No. 62/778,385, filed on Dec. 12, 2018.
(60) Provisional application No. 62/811,238, filed on Feb. 27, 2019.
(60) Provisional application No. 62/730,478, filed on Sep. 12, 2018.

(51) Int. Cl. C07K 16/24; A61K 9/00; A61P 17/04

(52) U.S. Cl. CPC C07K 16/245; A61P 17/04; A61K 9/0019

(57) ABSTRACT

Symptoms of atopic dermatitis in a human subject are reduced by administering to the subject a pharmaceutical composition that includes a pharmaceutically acceptable carrier and a therapeutically effective amount of an agent that selectively binds IL-1α.

### CROSS-REFERENCE TO RELATED APPLICATIONS

The present application claims the priority of U.S. provisional patent application Ser. No. 62/662,055, filed on Apr. 24, 2018, U.S. provisional patent application Ser. No. 62/730,478, filed on Sep. 12, 2018, U.S. provisional patent application Ser. No. 62/778,385, filed on Dec. 12, 2018, and U.S. provisional patent application Ser. No. 62/811,238, filed on Feb. 27, 2019.

### STATEMENT AS TO FEDERALLY SPONSORED RESEARCH

Not applicable.

### FIELD OF THE INVENTION

The invention relates generally to the fields of medicine, dermatology, and immunology. More particularly, the invention relates to the use of antibodies (Abs) which specifically bind interleukin-1α (IL-1α) to treat various symptoms of atopic dermatitis.

### BACKGROUND

Atopic dermatitis (AD), also known as eczema, is an inflammatory skin disease affecting as much as 20% of the population in western industrial societies. Chronic eczema in AD and particularly the associated pruritus can be a significant cause of morbidity and impact life quality. Disease pathogenesis is complex but ultimately converges on a pathological inflammatory process that disrupts the protective barrier function of the skin.

### SUMMARY

It was discovered that a monoclonal antibody (mAb) that specifically binds IL-1α is useful for treating the symptoms of AD.

Accordingly, disclosed herein are methods of reducing one or more symptoms of AD in a human subject. These methods can include the step of administering to the subject a pharmaceutical composition including a pharmaceutically acceptable carrier and an amount of an agent that selectively binds IL-1α effective to reduce to reduce a symptom of AD in the subject. The agent can be an anti-IL-1α antibody such as a monoclonal antibody (e.g., of the IgG1 isotype), a monoclonal antibody that includes a complementarity determining region of bermekimab (MABp1), or bermekimab (MABp1). The pharmaceutical composition can be administered to the subject by injection, subcutaneously, intravenously, intramuscularly, or intradermally. In the method, the dose can be at least 50 mg (e.g., at least 50, 75, 100, 150, 200, 300, 400, 500, 600, 700, or 800 mg). Preferably, at least 200 mg (e.g., 200, 300, 400, 500, 600, 700, or 800 mg) bermekimab is administered at least once a week (e.g., 1, 2, 3 times a week) by subcutaneous injection for at least 2 weeks (e.g., at least 2, 3, 4, 5, 6, 7, 8, 9, 10, 15, 20, 50 weeks) or until a symptom of AD is reduced or cleared.

It was also discovered that viscous, high mAb concentrations provided significantly improved (more than linear) mAb bioavailability. Accordingly, described herein are mAb formulations that include about 180, 200, 220, 240, 260, 280, 300, or more mAb per ml of the pharmaceutical composition, and mAb formulations that have a viscosity of at least about 20 cP (centipoise) at 25° C. (e.g., at least 19, 20, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 cP at 25° C.). Also described herein are pharmaceutical compositions containing a mAb at a concentration of about 200 mg/ml or more, pharmaceutical compositions containing a mAb that have a viscosity of at least about 20 cP at 25° C., and pharmaceutical compositions containing a mAb at a concentration of about 200 mg/ml or more and a viscosity of at least about 20 cP at 25° C. Further described herein, are methods of increasing the bioavailability of a mAb by increasing the concentration of the mAb to about 180, 200, 220, 240, 260, 280, 300, or more mAb per ml of the pharmaceutical composition and/or increasing viscosity of the mAb-containing pharmaceutical composition to at least about 20 cP (centipoise) at 25° C. (e.g., at least 19, 20, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 cP at 25° C.)

Unless otherwise defined, all technical terms used herein have the same meaning as commonly understood by one of ordinary skill in the art to which this invention belongs. Commonly understood definitions of biological terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of medical terms can be found in Stedman's Medical Dictionary, 27^{th} Edition, Lippincott, Williams & Wilkins, 2000.

As used herein, an “antibody” or “Ab” is an immunoglobulin (Ig), a solution of identical or heterogeneous Igs, or a mixture of Igs. An “Ab” can also refer to fragments and engineered versions of Igs such as Fab, Fab′, and F(ab′)₂ fragments; and scFv's, heteroconjugate Abs, and similar artificial molecules that employ Ig-derived CDRs to impart antigen specificity. A “monoclonal antibody” or “mAb” is an Ab expressed by one clonal B cell line or a population of Ab molecules that contains only one species of an antigen binding site capable of immunoreacting with a particular epitope of a particular antigen. A “polyclonal Ab” is a mixture of heterogeneous Abs. Typically, a polyclonal Ab will include myriad different Ab molecules which bind a particular antigen with at least some of the different Abs immunoreacting with a different epitope of the antigen. As used herein, a polyclonal Ab can be a mixture of two or more mAbs.

An “antigen-binding portion” of an Ab is contained within the variable region of the Fab portion of an Ab and is the portion of the Ab that confers antigen specificity to the Ab (i.e., typically the three-dimensional pocket formed by the CDRs of the heavy and light chains of the Ab). A “Fab portion” or “Fab region” is the proteolytic fragment of a papain-digested Ig that contains the antigen-binding portion of that Ig. A “non-Fab portion” is that portion of an Ab not within the Fab portion, e.g., an “Fc portion” or “Fc region.” A “constant region” of an Ab is that portion of the Ab outside of the variable region. Generally encompassed within the constant region is the “effector portion” of an Ab, which is the portion of an Ab that is responsible for binding other immune system components that facilitate the immune response. Thus, for example, the site on an Ab that binds complement components or Fc receptors (not via its antigen-binding portion) is an effector portion of that Ab.

When referring to a protein molecule such as an Ab, “purified” means separated from components that naturally accompany such molecules. Typically, an Ab or protein is purified when it is at least about 10% (e.g., 9%, 10%, 20%, 30% 40%, 50%, 60%, 70%, 80%, 90%, 95%, 98%, 99%, 99.9%, and 100%), by weight, free from the non-Ab proteins or other naturally-occurring organic molecules with which it is naturally associated. Purity can be measured by any appropriate method, e.g., column chromatography, polyacrylamide gel electrophoresis, or HPLC analysis. A chemically-synthesized protein or other recombinant protein produced in a cell type other than the cell type in which it naturally occurs is “purified.”

By “bind”, “binds”, or “reacts with” is meant that one molecule recognizes and adheres to a particular second molecule in a sample, but does not substantially recognize or adhere to other molecules in the sample. Generally, an Ab that “specifically binds” another molecule has a K_{d} greater than about 10⁵, 10⁶, 10⁷, 10⁸, 10⁹, 10¹⁰, 10¹¹, or 10¹² liters/mole for that other molecule. An Ab that “selectively binds” a first molecule specifically binds the first molecule at a first epitope but does not specifically bind other molecules that do not have the first epitope. For example, an Ab which selectively binds IL-1 alpha specifically binds an epitope on IL-1 alpha but does not specifically bind IL-1beta (which does not have the epitope).

A “therapeutically effective amount” is an amount which is capable of producing a medically desirable effect in a treated animal or human (e.g., amelioration or prevention of a disease or symptom of a disease).

As used herein, “about” means +/−20 percent.

Although methods and materials similar or equivalent to those described herein can be used in the practice or testing of the present invention, suitable methods and materials are described below. All applications and publications mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is a flow chart showing an overview of the treatment protocol of the clinical study described in the Examples section below.

FIG. 2 is a chart showing the baseline characteristics of the study populations which participated in the clinical study described in the Examples section below.

FIG. 3 is a chart listing the adverse events observed in the clinical study described in the Examples section below.

FIG. 4 is a study calendar of the clinical study described in the Examples section below.

FIG. 5 is a graph showing the mean improvement in EASI score observed in the clinical study described in the Examples section below.

FIG. 6 is a graph showing the percent of subjects achieving EASI-75 in the clinical study described in the Examples section below.

FIG. 7 is a graph comparing the percent of subjects achieving EASI-75 score observed in the clinical study described in the Examples section below versus data published for dupilumab.

FIG. 8 is a graph showing the mean improvement in SCORAD observed in the clinical study described in the Examples section below.

FIG. 9 is a graph comparing the mean improvement in SCORAD observed in the clinical study described in the Examples section below between the 200 mg and 400 mg groups.

FIG. 10 is a graph comparing the percent improvement in SCORAD observed in the clinical study described in the Examples section below versus data published for dupilumab.

FIG. 11 is a graph showing the mean improvement in GISS observed in the clinical study described in the Examples section below.

FIG. 12 is a graph comparing the percent improvement in GISS observed in the clinical study described in the Examples section below versus data published for dupilumab.

FIG. 13 is a graph showing the percent of subjects achieving at least a 2 point reduction in IGA in the clinical study described in the Examples section below.

FIG. 14 is a graph comparing the percent of subject achieving at least a 4 point reduction in IGA and a final IGA score of 0 or 1 in the clinical study described in the Examples section below versus data published for dupilumab.

FIG. 15 is a graph showing the mean improvement in DLQI score observed in the clinical study described in the Examples section below.

FIG. 16 is a graph comparing the mean point reduction in DLQI for subjects in the clinical study described in the Examples section below versus data published for dupilumab.

FIG. 17 is a graph showing the mean improvement in POEM score observed in the clinical study described in the Examples section below.

FIG. 18 is a graph comparing the mean improvement in POEM score observed at 4 weeks in the clinical study described in the Examples section below between the 200 mg and 400 mg groups.

FIG. 19 is a graph showing the mean point reduction in POEM score for subjects in the clinical study described in the Examples section below.

FIG. 20 is a graph comparing the mean point reduction in POEM score for subjects in the clinical study described in the Examples section below versus data published for dupilumab.

FIG. 21 is a graph showing the mean improvement in HADS score for anxiety and depression observed in the clinical study described in the Examples section below.

FIG. 22 is a graph showing the mean point reduction in HADS depression score for subjects in the clinical study described in the Examples section below.

FIG. 23 is a graph showing the mean point reduction in HADS combined score for subjects in the clinical study described in the Examples section below.

FIG. 24 is a graph comparing the mean point reduction in HADS combined score for subjects in the clinical study described in the Examples section below versus data published for dupilumab.

FIG. 25 is a graph showing the percent of subjects achieving at least a 4 point reduction in NRS Worst Itch score observed in the clinical study described in the Examples section below.

FIG. 26 is a graph showing the percent of subjects achieving at least a 4 point reduction in NRS Overall Itch score observed in the clinical study described in the Examples section below.

FIG. 27 is a graph comparing the percent of subjects achieving at least a 4 point reduction in week 4 NRS Worst Itch score observed in the clinical study described in the Examples section below versus data published for dupilumab.

FIG. 28 is a graph showing the percent of subjects achieving at least a 4 point reduction in NRS Pain score observed in the clinical study described in the Examples section below.

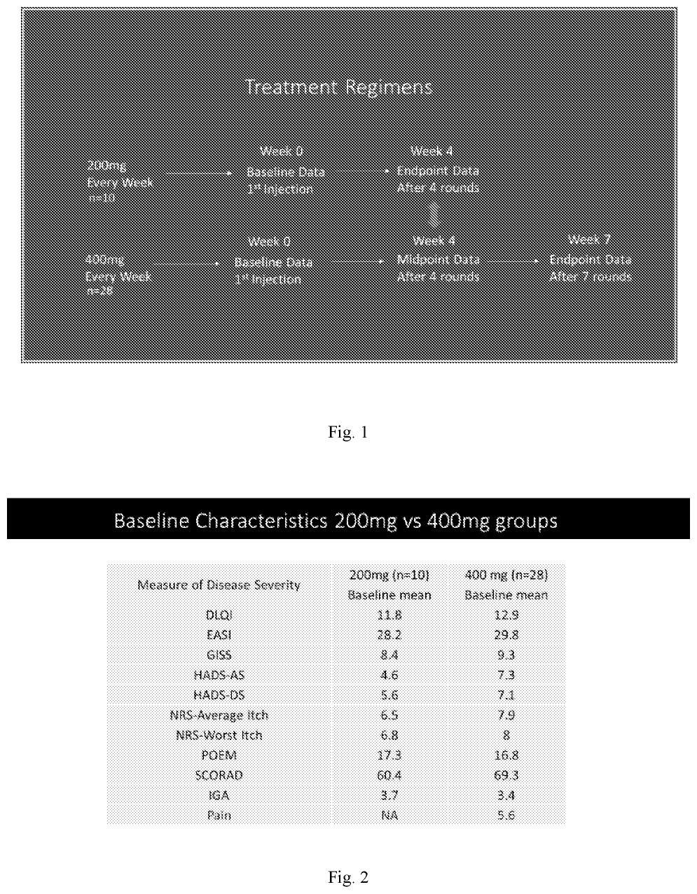

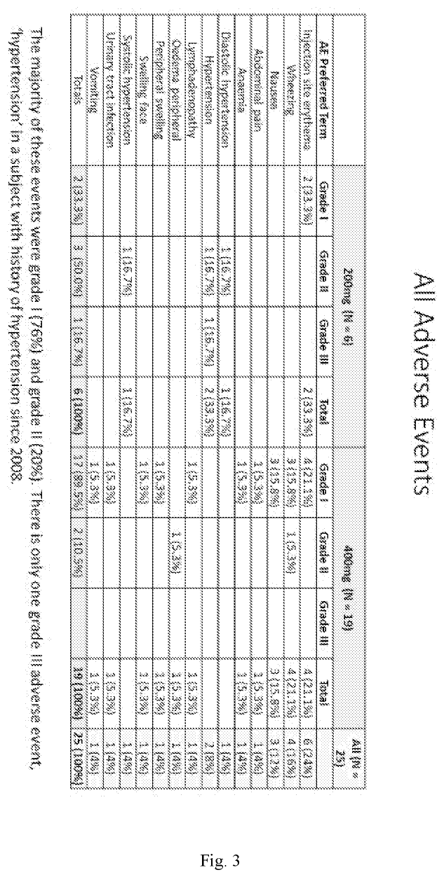

### DETAILED DESCRIPTION

Described herein are compositions and methods for reducing a symptom of AD in a subject. The below described preferred embodiments illustrate adaptation of these compositions and methods. Nonetheless, from the description of these embodiments, other aspects of the invention can be made and/or practiced based on the description provided below.

### General Methodology

Methods involving conventional immunological and molecular biological techniques are described herein Immunological methods (for example, assays for detection and localization of antigen-Ab complexes, immunoprecipitation, immunoblotting, and the like) are generally known in the art and described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. Ab methods are described in Handbook of Therapeutic Abs, Dubel, S., ed., Wiley-VCH, 2007. General methods of medical treatment are described in McPhee and Papadakis, Current Medical Diagnosis and Treatment 2010, 49^{th} Edition, McGraw-Hill Medical, 2010; and Fauci et al., Harrison's Principles of Internal Medicine, 17th Edition, McGraw-Hill Professional, 2008. Methods in dermatology are described in James et al., Andrews' Diseases of the Skin: Clinical Dermatology—Expert Consult, 11^{th} Ed., Saunders, 2011; and Burns et al., Rook's Textbook of Dermatology, 8^{th} Ed., Wiley-Blackwell, 2010.

### Treatment

The compositions and methods described herein are useful for treating a symptom of AD (e.g., erythema, excoriation, papulation, infiltration, lichenification, itching, pain, oozing, crusting, swelling, bleeding, scratch marks, flaking, and sleep loss). Successful treatment of AD can be evaluated according to established assays known in the art. These include: Eczema Area and Severity Index Score (EASI) which is used to assess the severity and extent of AD with respect to erythema, excoriation, infiltration and lichenification at 4 anatomic sites of the body: lower and upper extremities, trunk and head; Investigator's Global Assessment (IGA) which is used to assess disease severity and clinical response using a 5-point scale by ranking the extent of erythema and papulation/infiltration: 0=clear; 1=almost clear; 2=mild; 3=moderate; 4=severe; Pruritus numerical rating system (NRS) which captures the intensity of a patient's itch and pain over a 24-hour period; SCORing Atopic Dermatitis (SCORAD) which is used to assess eczema by clinical presentation (redness, swelling, oozing/crusting, scratch marks, lichenification, and dryness) and patient reported symptoms (itch, sleeplessness); Patient Oriented Eczema Measure (POEM) which is a patient reported quality of life outcome measure based on a questionnaire to determine disease symptoms, including bleeding, cracking, dryness, flaking, itching, sleep loss and weeping/oozing; and Global Individual Signs Score (GISS) which assesses AD lesions for erythema, excoriations, lichenification and edema/papulation. A reduction in a symptom of AD includes a reduction of at least 8 points in a patient's EASI score, a reduction of at least 1 point in a patient's IGA score; a reduction of at least 2 points in a patient's NRS score (itch or pain); a reduction of at least 10 points in a patient's SCORAD score; a reduction of at least 3 points in a patient's POEM score; and a reduction of at least 2 points in a patient's total GISS score.

The mammalian subject might be any that suffers from AD including, human beings. Human subjects might be male, female, adults, children, seniors (65 and older), and those with other diseases. Particularly preferred subjects are those whose disease has progressed or failed to respond after treatment with other anti-inflammatory agents such as topical corticosteroids, topical calcineurin inhibitors, oral corticosteroids, dupilumab, nemolizumab, and phototherapy. Subjects who have developed a human anti-human antibody response due to prior administration of therapeutic antibodies are preferred when the anti-IL-1α Ab is a true human Ab (e.g., one with all V regions naturally expressed in a human subject) such as bermekimab (MABp1).

### Antibodies and Other Agents that Target IL-1α

Any suitable type of Ab that specifically binds IL-1α and reduces a characteristic of AD in a subject might be used in the methods described herein. For example, the anti-IL-1α Ab used might be mAb, a polyclonal Ab, a mixture of mAbs, or an Ab fragment or engineered Ab-like molecule such as an scFv. The Ka of the Ab is preferably at least 1×10⁹ M⁻¹ or greater (e.g., greater than 9×10¹⁰ M⁻¹, 8×10¹⁰ M⁻¹, 7×10¹⁰ M⁻¹, 6×10¹⁰ M⁻¹, 5×10¹⁰ M⁻¹, 4×10¹⁰ M⁻¹, 3×10¹⁰ M⁻¹, 2×10¹⁰ M⁻¹, or 1×10¹⁰ M⁻¹). In a preferred embodiment, the Ab is a fully human mAb that includes (i) an antigen-binding variable region that exhibits very high binding affinity (e.g., at least nano or picomolar) for human IL-1α and (ii) a constant region. The human Ab is preferably an IgG1, although it might be of a different isotype such as IgM, IgA, or IgE, or subclass such as IgG2, IgG3, or IgG4. One example of a particularly useful mAb is bermekimab (MABp1), an IL-1α-specific IgG1 mAb described in U.S. Pat. No. 8,034,337. Other useful mAbs are those that include at least one but preferably all the CDRs of bermekimab, those that neutralize IL-1α (e.g., those that prevent IL-1α from binding an IL-1α receptor), and those that compete for binding to IL-1α with bermekimab (e.g., by competition ligand-receptor interaction assay).

Because B lymphocytes which express Ig specific for human IL-1c occur naturally in human beings, a presently preferred method for raising mAbs is to first isolate such a B lymphocyte from a subject and then immortalize it so that it can be continuously replicated in culture. Subjects lacking large numbers of naturally occurring B lymphocytes which express Ig specific for human IL-1α may be immunized with one or more human IL-1α antigens to increase the number of such B lymphocytes. Human mAbs are prepared by immortalizing a human Ab secreting cell (e.g., a human plasma cell). See, e.g., U.S. Pat. No. 4,634,664.

In an exemplary method, one or more (e.g., 5, 10, 25, 50, 100, 1000, or more) human subjects are screened for the presence of such human IL-1α-specific Ab in their blood. Those subjects that express the desired Ab can then be used as B lymphocyte donors. In one possible method, peripheral blood is obtained from a human donor that possesses B lymphocytes that express human IL-1α-specific Ab. Such B lymphocytes are then isolated from the blood sample, e.g., by cells sorting (e.g., fluorescence activated cell sorting, “FACS”; or magnetic bead cell sorting) to select B lymphocytes expressing human IL-1α-specific Ig. These cells can then be immortalized by viral transformation (e.g., using EBV) or by fusion to another immortalized cell such as a human myeloma according to known techniques. The B lymphocytes within this population that express Ig specific for human IL-1α can then be isolated by limiting dilution methods (e.g., cells in wells of a microtiter plate that are positive for Ig specific for human IL-1α are selected and subcultured, and the process repeated until a desired clonal line can be isolated). See, e.g., Goding, MAbs: Principles and Practice, pp. 59-103, Academic Press, 1986. Those clonal cell lines that express Ig having at least nanomolar or picomolar binding affinities for human IL-1α are preferred. MAbs secreted by these clonal cell lines can be purified from the culture medium or a bodily fluid (e.g., ascites) by conventional Ig purification procedures such as salt cuts, size exclusion, ion exchange separation, and affinity chromatography.

Although immortalized B lymphocytes might be used in in vitro cultures to directly produce mAbs, in certain cases it might be desirable to use heterologous expression systems to produce mAbs. See, e.g., the methods described in U.S. patent application Ser. No. 11/754,899. For example, the genes encoding an mAb specific for human IL-1α might be cloned and introduced into an expression vector (e.g., a plasmid-based expression vector) for expression in a heterologous host cell (e.g., CHO cells, COS cells, myeloma cells, and E. coli cells). Because Igs include heavy (H) and light (L) chains in an H₂L₂ configuration, the genes encoding each may be separately isolated and expressed in different vectors.

Although generally less preferred due to the greater likelihood that a subject will develop an anti-Ab response, chimeric mAbs (e.g., “humanized” mAbs), which are antigen-binding molecules having different portions derived from different animal species (e.g., variable region of a mouse Ig fused to the constant region of a human Ig), might be used in the methods described herein. Such chimeric Abs can be prepared by methods known in the art. See, e.g., Morrison et al., Proc. Nat'l. Acad. Sci. USA, 81:6851, 1984; Neuberger et al., Nature, 312:604, 1984; Takeda et al., Nature, 314:452, 1984. Similarly, Abs can be humanized by methods known in the art. For example, mAbs with a desired binding specificity can be humanized by various vendors or as described in U.S. Pat. No. 5,693,762; 5,530,101; or 5,585,089.

The mAbs described herein might be affinity matured to enhance or otherwise alter their binding specificity by known methods such as VH and VL domain shuffling (Marks et al. Bio/Technology 10:779-783, 1992), random mutagenesis of the hypervariable regions (HVRs) and/or framework residues (Barbas et al. Proc Nat. Acad. Sci. USA 91:3809-3813, 1994; Schier et al. Gene 169:147-155, 1995; Yelton et al. J. Immunol. 155:1994-2004, 1995; Jackson et al., J. Immunol. 154(7):3310-9, 1995; and Hawkins et al, J. Mol. Biol. 226:889-896, 1992 Amino acid sequence variants of an Ab may be prepared by introducing appropriate changes into the nucleotide sequence encoding the Ab. In addition, modifications to nucleic acid sequences encoding mAbs might be altered (e.g., without changing the amino acid sequence of the mAb) for enhancing production of the mAb in certain expression systems (e.g., intron elimination and/or codon optimization for a given expression system). The mAbs described herein can also be modified by conjugation to another protein (e.g., another mAb) or non-protein molecule. For example, a mAb might be conjugated to a water soluble polymer such as polyethylene glycol or a carbon nanotube (See, e.g., Kam et al., Proc. Natl. Acad. Sci. USA 102: 11600-11605, 2005). See, U.S. patent application Ser. No. 11/754,899.

Preferably, to ensure that high titers of human IL-1α-specific mAb can be administered to a subject with minimal adverse effects, the mAb compositions should be at least 0.5, 1, 2, 3, 4, 5, 6, 7, 8, 9, 10, 11, 12, 13, 14, 15, 20, 25, 30, 35, 40, 45, 50, 60, 70, 80, 90, 95, 96, 97, 98, 99, 99.9 or more percent by weight pure (excluding any excipients). The mAb compositions might include only a single type of mAb (i.e., one produced from a single clonal B lymphocyte line) or might include a mixture of two or more (e.g., 2, 3, 4, 5, 6, 7, 8, 9, 10 or more) different types of mAbs.

While the IL-1α specific Abs described above are preferred for use in the methods described herein, in some cases, other agents that specifically target IL-1α might be used so long as their administration leads to improvement of a characteristic AD. Because bermelomab has been shown to block the action of IL-1α by preventing its interaction with the IL-1 receptor (IL-1R1), based on this mechanism of action in treating AD, other Abs or non-Ab agents that also block IL-1α from interacting with IL-1R1 could also be used to reduce a symptom of AD (e.g., other anti-IL-1α Abs or anti-IL-1R1 Abs which block IL-1α from interacting with IL-1R1). These Abs can be made according to the methods described above. Non-Ab agents might include vaccines that cause the production of anti-IL-1α Abs which block IL-1α from interacting with IL-1R1, proteins or peptides that bind IL-1α and block IL-1α from interacting with IL-1R1, and small organic molecules which specifically target IL-1α and block IL-1α from interacting with IL-1R1. Those that do not specifically bind other agents that specifically target IL-1β are preferred. Whether a particular agent is able to treat one or more symptoms of AD in a subject can be determined by the methods described in the Examples section below and those that are known in the art.

### Pharmaceutical Compositions and Methods

The anti-IL-1α Ab compositions (and other agents that specifically target IL-1α) may be administered to animals or humans in pharmaceutically acceptable carriers (e.g., sterile saline), that are selected on the basis of mode and route of administration and standard pharmaceutical practice. A list of pharmaceutically acceptable carriers, as well as pharmaceutical formulations, can be found in Remington's Pharmaceutical Sciences, a standard text in this field, and in USP/NF. Other substances may be added to the compositions and other steps taken to stabilize and/or preserve the compositions, and/or to facilitate their administration to a subject.

For example, the Ab compositions might be lyophilized (see Draber et al., J. Immunol. Methods. 181:37, 1995; and PCT/US90/01383); dissolved in a solution including sodium and chloride ions; dissolved in a solution including one or more stabilizing agents such as albumin, glucose, maltose, sucrose, sorbitol, polyethylene glycol, and glycine; filtered (e.g., using a 0.45 and/or 0.2 micron filter); contacted with beta-propiolactone; and/or dissolved in a solution including a microbicide (e.g., a detergent, an organic solvent, and a mixture of a detergent and organic solvent.

The Ab compositions may be administered to animals or humans by any suitable technique. Typically, such administration will be parenteral (e.g., intravenous, subcutaneous, intramuscular, or intraperitoneal introduction). The compositions may also be administered directly to the target site (e.g., the skin) by, for example, topical application. Other methods of delivery, e.g., liposomal delivery or diffusion from a device impregnated with the composition, are known in the art. The composition may be administered in a single bolus, multiple injections, or by continuous infusion (e.g., intravenously or by peritoneal dialysis).

A therapeutically effective amount is an amount which is capable of producing a medically desirable result in a treated animal or human. An effective amount of anti-IL-1α Ab compositions is an amount which shows clinical efficacy in patients as measured by the improvement in one or more symptoms of AD. As is well known in the medical arts, dosage for any one animal or human depends on many factors, including the subject's size, body surface area, age, the particular composition to be administered, sex, time and route of administration, general health, and other drugs being administered concurrently. Preferred doses range from about 3 to 20 (e.g., 2, 3, 4, 5, 6, 7, 8, 9, 10, 11, 12, 13, 14, 15, 16, 17, 18, 19, 20, 21, or 22) mg/kg body weight. In some cases, a single dose may be effective at resolving a symptom of AD. In other cases, doses may be given repeatedly, e.g., semi-weekly, weekly, bi-weekly, tri-weekly, semi-monthly, once every three weeks, monthly, bi-monthly, or as needed (if the symptom of AD recurs or to prevent recurrence of AD symptoms once resolved).

The mAbs described herein as well as other mAbs can include about 180, 200, 220, 240, 260, 280, 300, or more mAb per ml of the pharmaceutical composition, and/or can be formulated as a liquid composition that have a viscosity of at least about 20 cP (centipoise) at 25° C. (e.g., at least 19, 20, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 cP at 25° C.). The isoelectric points (pI) of such antibodies can be about 6, 6.5., 7, 7.5, 8, 8.5, 9, 9.5, or 10 (e.g., as determined by imaged capillary isoelectric focusing). Examples of other mAbs include those that target TNF-α, IL-O, IL-2, IL-4, IL-5, IL-6, IL-12, IL-13, IL-17A, IL-22, IL-31, IL-33, IFN-γ, and GM-CSF. Other mAbs also include: Examples of antibodies include, without limitation, Infliximab, Bevacizumab, Ranibizumab, Cetuximab, Ranibizumab, Palivizumab, Abagovomab, Abciximab, Actoxumab, Adalimumab, Afelimomab, Afutuzumab, Alacizumab, Alacizumab pegol, ALD518, Alemtuzumab, Alirocumab, Alemtuzumab, Altumomab, Amatuximab, Anatumomab mafenatox, Anrukinzumab, Apolizumab, Arcitumomab, Aselizumab, Altinumab, Atlizumab, Atorolimiumab, tocilizumab, Bapineuzumab, Basiliximab, Bavituximab, Bectumomab, Belimumab, Benralizumab, Bertilimumab, Besilesomab, Bevacizumab, Bezlotoxumab, Biciromab, Bivatuzumab, Bivatuzumab mertansine, Blinatumomab, Blosozumab, Brentuximab vedotin, Briakinumab, Brodalumab, Canakinumab, Cantuzumab mertansine, Cantuzumab mertansine, Caplacizumab, Capromab pendetide, Carlumab, Catumaxomab, CC49, Cedelizumab, Certolizumab pegol, Cetuximab, Citatuzumab bogatox, Cixutumumab, Clazakizumab, Clenoliximab, Clivatuzumab tetraxetan, Conatumumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Ecromeximab, Eculizumab, Edobacomab, Edrecolomab, Efalizumab, Efungumab, Elotuzumab, Elsilimomab, Enavatuzumab, Enlimomab pegol, Enokizumab, Enokizumab, Enoticumab, Enoticumab, Ensituximab, Epitumomab cituxetan, Epratuzumab, Erlizumab, Ertumaxomab, Etaracizumab, Etrolizumab, Exbivirumab, Exbivirumab, Fanolesomab, Faralimomab, Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lebrikizumab, Lemalesomab, Lerdelimumab, Lexatumumab, Libivirumab, Ligelizumab, Lintuzumab, Lirilumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pintumomab, Placulumab, Ponezumab, Priliximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Tefibazumab, Telimomab aritox, Tenatumomab, Teneliximab, Teplizumab, Teprotumumab, TGN1412, tremelimumab, Ticilimumab, Tildrakizumab, Tigatuzumab, TNX-650, Tocilizumab, Torahzumab, Tositumomab, Tralokinumab, Trastuzumab, TRBS07, Tregalizumab, Tremelimumab, Tucotuzumab celmoleukin, Tuvirumab, Ublituximab, Urelumab, Urtoxazumab, Ustekinumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab and Zolimomab aritox.

### EXAMPLES

### Example 1—Open Label Study of Subcutaneous Bermekimab (MABp1) Administration in Two Dose Cohorts for Moderate to Severe Atopic Dermatitis

Group A (n=9): patients receive a total of 4×200 mg subcutaneous injections of bermekimab. Dosing is performed weekly from visit 1 to visit 4. Group B (n=20): patients receive a total of 8×400 mg subcutaneous injections of bermekimab. Dosing will occur weekly from visit 1 to visit 8.

Inclusion Criteria:
Subjects are included in the study if they meet all of the following criteria:
Written informed consent provided by the patient
Age 18 years or older
Chronic Atopic Dermatitis present for at least 3 years
Disease is not responsive to topical medications, or for whom topical treatments are not indicated or desired
Willing and able to comply with all clinic visits and study-related procedure
EASI score ≥16 at screening and baseline visits
IGA score ≥3 at screening and baseline visits
≥10% body surface area (BSA) of AD involvement at screening and baseline visits
Documented recent history (within 6 months before the screening visit) of inadequate response to treatment with topical medications or for whom topical treatments are otherwise medically inadvisable or undesired

Exclusion Criteria:
Subjects with ANY of the following will be excluded from the study:
Treatment with an investigational drug within 8 weeks of baseline visit
Having received the following treatments within 4 weeks before the baseline visit, or any condition that, in the opinion of the investigator, is likely to require such treatment(s) during the first 4 weeks of study treatment:
Immunosuppressive/immunomodulating drugs (eg, systemic corticosteroids, cyclosporine, mycophenolate-mofetil, IFN-γ, Janus kinase inhibitors, azathioprine, methotrexate, etc.)
Phototherapy for AD
Treatment with topical corticosteroids (TCS) or topical calcineurin inhibitors (TCI) within 1 week before the baseline visit
Initiation of treatment during the screening period with prescription moisturizers or moisturizers containing additives such as ceramide, hyaluronic acid, urea, or filaggrin degradation products during the screening period (patients may continue using stable doses of such moisturizers if initiated before the screening visit)
Regular use (more than 2 visits per week) of a tanning booth/parlor within 4 weeks of the screening visit
History of severe allergic or anaphylactic reactions to monoclonal antibodies
Administration of any live (attenuated) vaccine within 4 weeks prior to the baseline
Any history of dysplasia or history of malignancy (including lymphoma and leukemia) other than a successfully treated non-metastatic cutaneous squamous cell carcinoma, basal cell carcinoma or localized carcinoma in situ of the cervix
Active chronic or acute infection requiring treatment with systemic antibiotics, antivirals, antiparasitics, antiprotozoals, or antifungals within 2 weeks before the baseline visit, or superficial skin infections within 1 week before the baseline visit. NOTE: patients may be rescreened after infection resolves
Known or suspected history of immunosuppression, including history of invasive opportunistic infections (eg, tuberculosis [TB], histoplasmosis, listeriosis, coccidioidomycosis, pneumocystosis, aspergillosis) despite infection resolution: or unusually frequent, recurrent, or prolonged infections, per investigator judgment
History of human immunodeficiency virus (HIV) infection or positive HIV serology at screening
Positive with hepatitis B surface antigen (HBsAg) or hepatitis C antibody at the screening visit
Presence of skin comorbidities that may interfere with study assessments
Severe concomitant illness(es) that, in the investigator's judgment, would adversely affect the patient's participation in the study. Examples include, but are not limited to, patients with short life expectancy, patients with uncontrolled diabetes (HbA1c≥9%), patients with cardiovascular conditions (eg, stage III or IV cardiac failure according to the New York Heart Association classification), severe renal conditions (eg, patients on dialysis), hepatobiliary conditions (eg, Child-Pugh class B or C), neurological conditions (eg, demyelinating diseases), active major autoimmune diseases (eg, lupus, inflammatory bowel disease, rheumatoid arthritis, etc.), other severe endocrinological, gastrointestinal, metabolic, pulmonary or lymphatic diseases. The specific justification for patients excluded under this criterion will be noted in study documents (chart notes, case report forms [CRFs], etc.)
Pregnant or breastfeeding women, or women planning to become pregnant or breastfeed during the study
Where relevant, women unwilling to use adequate birth control

Drug Product Description

One dosage form is a sterile liquid formulation of 100 mg/mL bermekimab in a stabilizing isotonic subcutaneous formulation buffer at pH 6.2-6.5. Each 2-mL Type I borosilicate glass serum vial contains 2 mL of the formulation and is sealed with a 13-mm Daikyo Flurotec butyl rubber stopper and flip-off aluminum seal. The exact composition of the Drug Product is shown in Table 1 below.

TABLE 1
Composition of Drug Product [100 mg/mL]

| | Ingredient | | | | Amount per |
|---|---|---|---|---|---|
| Ingredient | Function | Grade | Manufacturer | Concentration | 2 mL vial |
| Bermekimab | Active | GMP | XBiotech | 100 mg/mL | 200 mg |
| Antibody | Substance | | USA Inc. | | |
| Trehalose | Tonacity | GMP, | Ferro- | 60 mg/mL | 120 mg |
| Dihydrate | | USP/NF, | Pfanstiehl | | |
| | | EP, low | (USA) | | |
| | | endotoxin | | | |
| Sodium | pH | GMP, EP, | J T Baker | 12 mg/mL | 24 mg |
| Phosphate | buffering | USP | (USA) | | |
| Dibasic | capacity | | | | |
| Citric Acid | pH | GMP, EP, | Fisher | 2 mg/mL | 4 mg |
| Monohydrate | buffering | USP, BP, | (USA) | | |
| | capacity | JP | | | |
| Water for | Solvent | GMP, EP, | Irvine | q.s. | q.s. |
| Injection | | USP | Scientific | | |
| | | | (USA) | | |
| Phosphoric | pH | GMP, EP, | J T Baker | pH | pH |
| Acid | adjustment | USP | (USA) | adjustment | adjustment |
| Sodium | pH | GMP, EP, | J T Baker | pH | pH |
| Hydroxide, | adjustment | USP | (USA) | adjustment | adjustment |

The other dosage form used is a sterile liquid formulation of 200 mg/mL bermekimab in a stabilizing isotonic subcutaneous formulation buffer at pH 6.2-6.5. See Table 2 below. The drug product is packaged in pre-filled syringes. The pre-filled syringes used are OMPI EZ-Fill Nexa, 2.25 mL 27G ½ needle, or a comparable alternative. The barrel of the syringe is clear glass borosilicate type 1 with AISI 304 stainless steel thin wall needle containing 2 mL of the formulation and is sealed with West 1-3 mL Novapure piston (plunger) with Flurotec coating.

TABLE 2
Composition Drug Product [200 mg/mL]

| | Ingredient | | | | Amount per |
|---|---|---|---|---|---|
| Ingredient | Function | Grade | Manufacturer | Concentration | 2 mL syringe |
| Bermekimab | Active | GMP | XBiotech | 200 mg/mL | 400 mg |
| Antibody | Substance | | USA Inc. | | |
| Trehalose | Tonicity | GMP, | Ferro- | 60 mg/mL | 120 mg |
| Dihydrate | | USP/NF, | Pfanstiehl | | |
| | | EP, low | (USA) | | |
| | | endotoxin | | | |
| Sodium | pH | GMP, EP, | J T Baker | 12 mg/mL | 24 mg |
| Phosphate | Buffering | USP | (USA) | | |
| Dibasic | Capacity | | | | |
| Citric Acid | pH | GMP, EP, | Fisher | 2 mg/mL | 4 mg |
| Monohydrate | Buffering | USP, BP, | (USA) | | |
| | Capacity | JP | | | |
| Water for | Solvent | GMP, EP, | Irvine | q.s. | q.s. |
| Injection | | USP | Scientific | | |
| | | | (USA) | | |
| Phosphoric | pH | GMP, EP, | J T Baker | pH | pH |
| Acid | adjustment | USP | (USA) | adjustment | adjustment |
| Sodium | pH | GMP, EP, | J T Baker | pH | pH |
| Hydroxide, | adjustment | USP | (USA) | adjustment | adjustment |

Method of Administration: The dose of bermekimab for Group A is 200 mg (2 ml of the 100 mg/ml formulation) and for Group B is 400 mg (2 ml of the 200 mg/ml formulation) administered weekly by subcutaneous injection.

Study Design and Objectives.

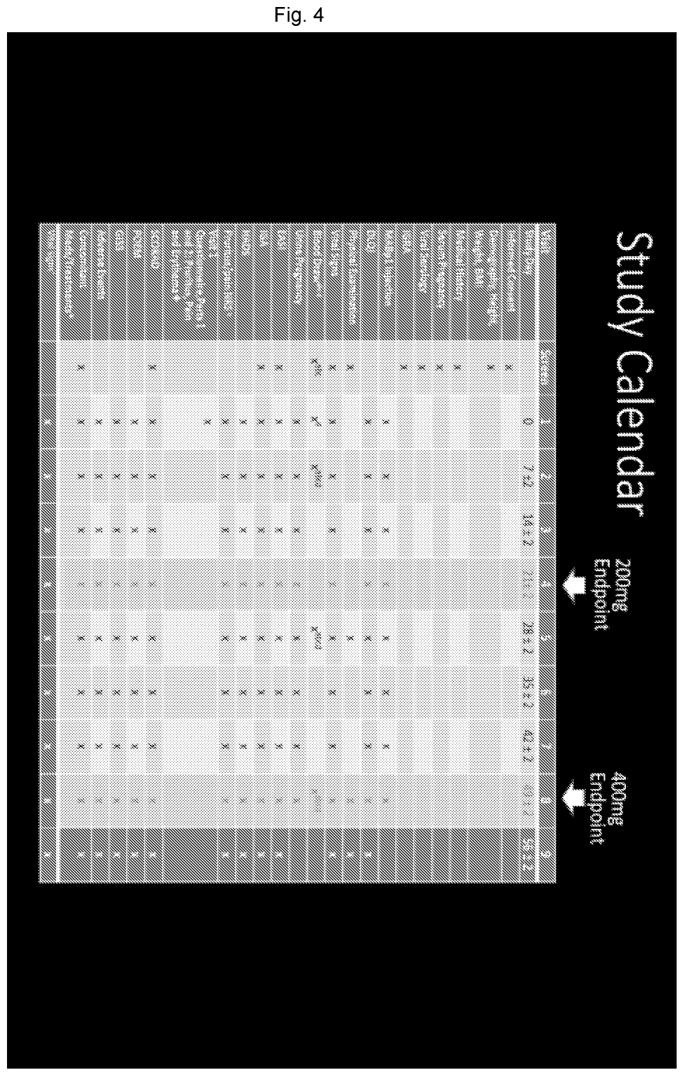

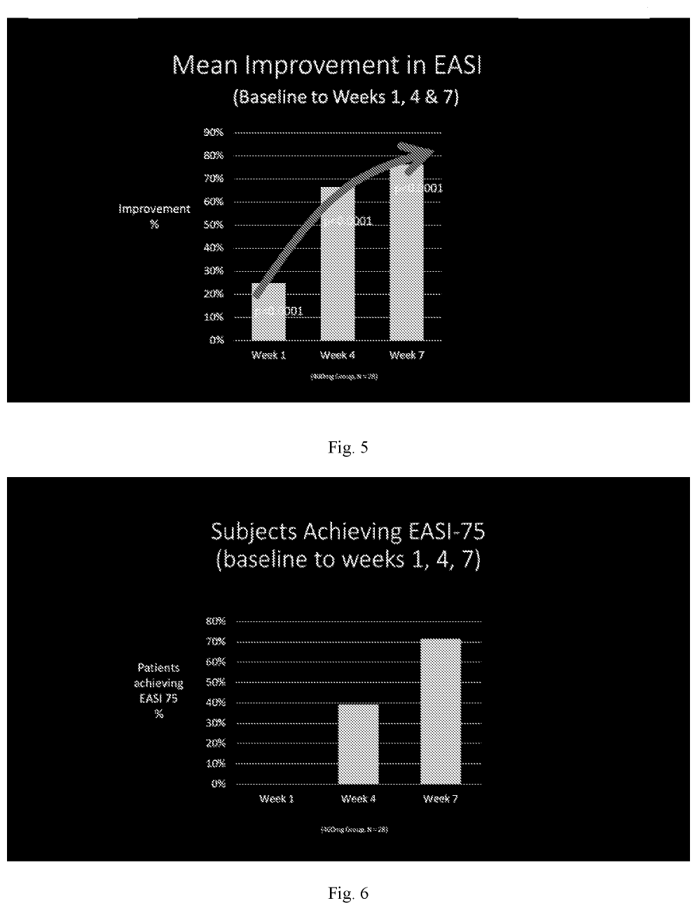

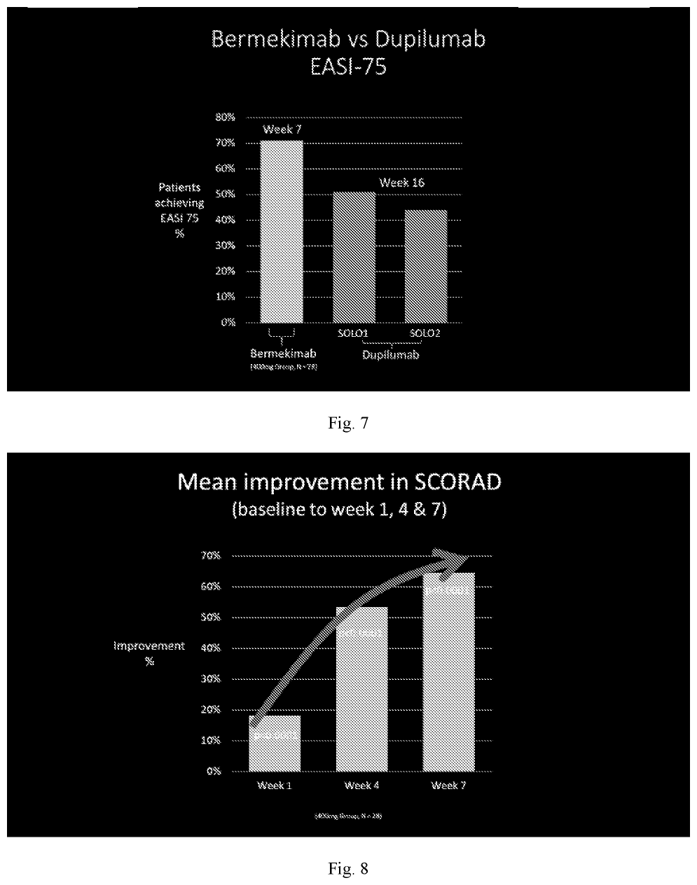

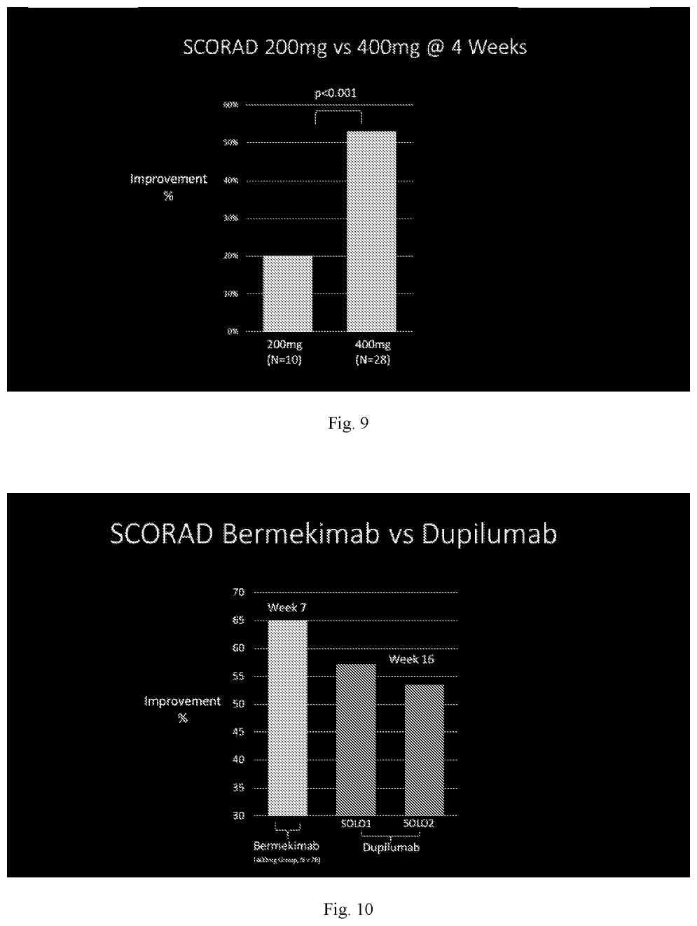

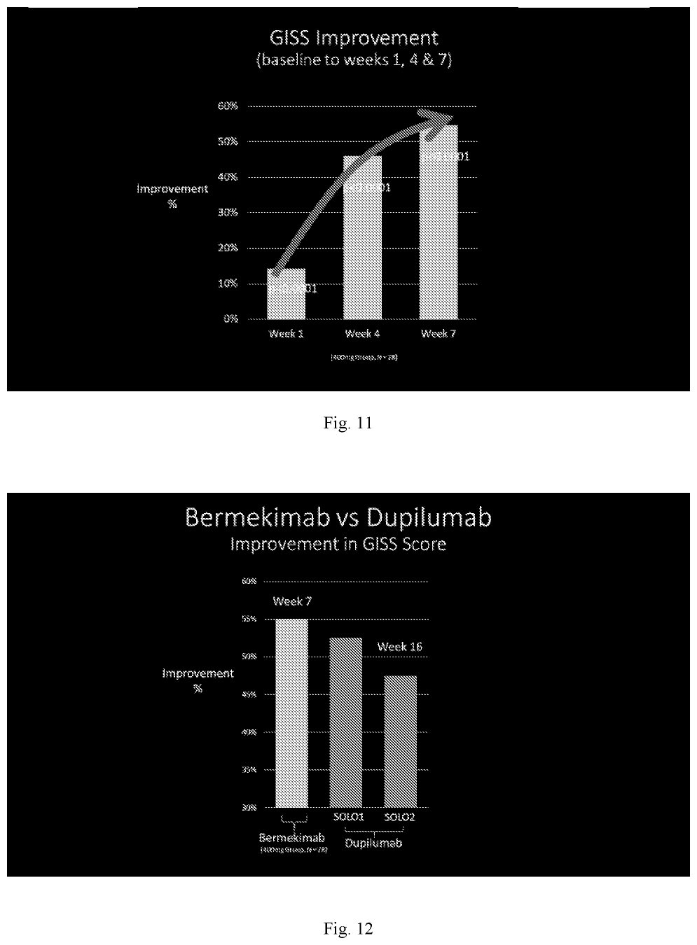

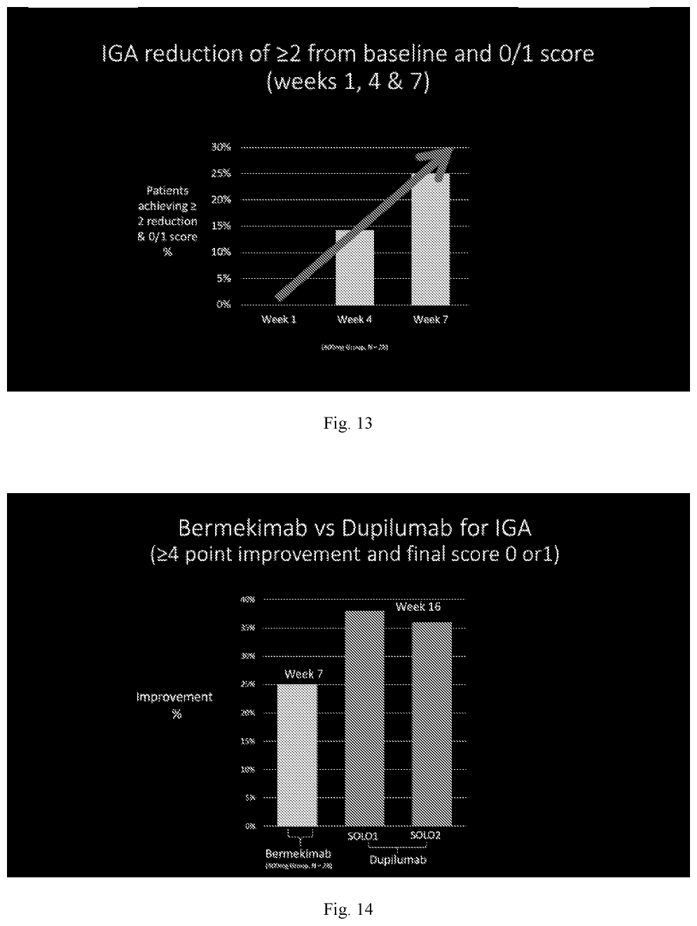

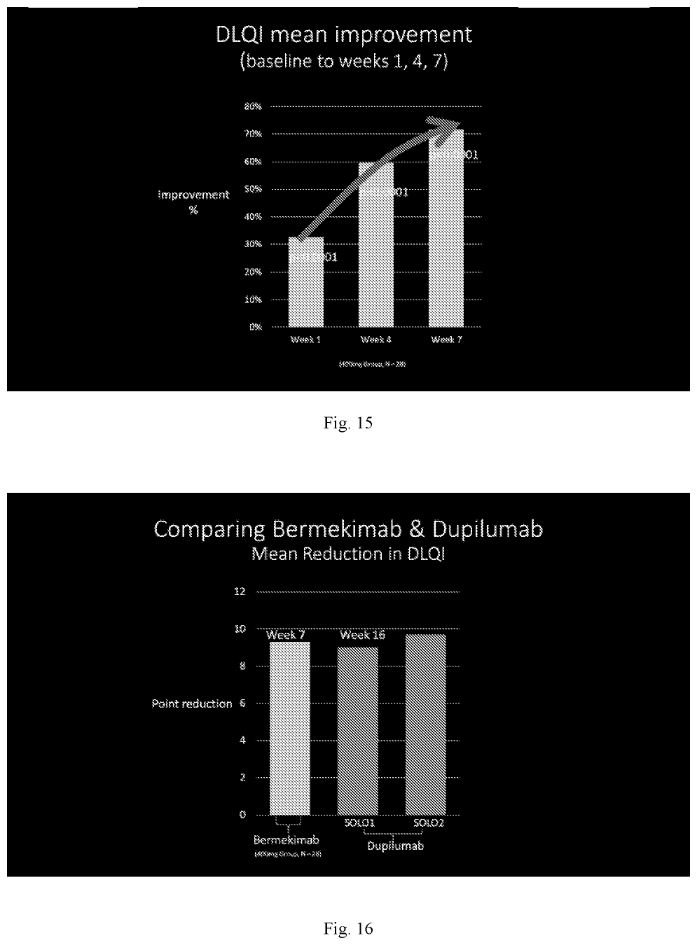

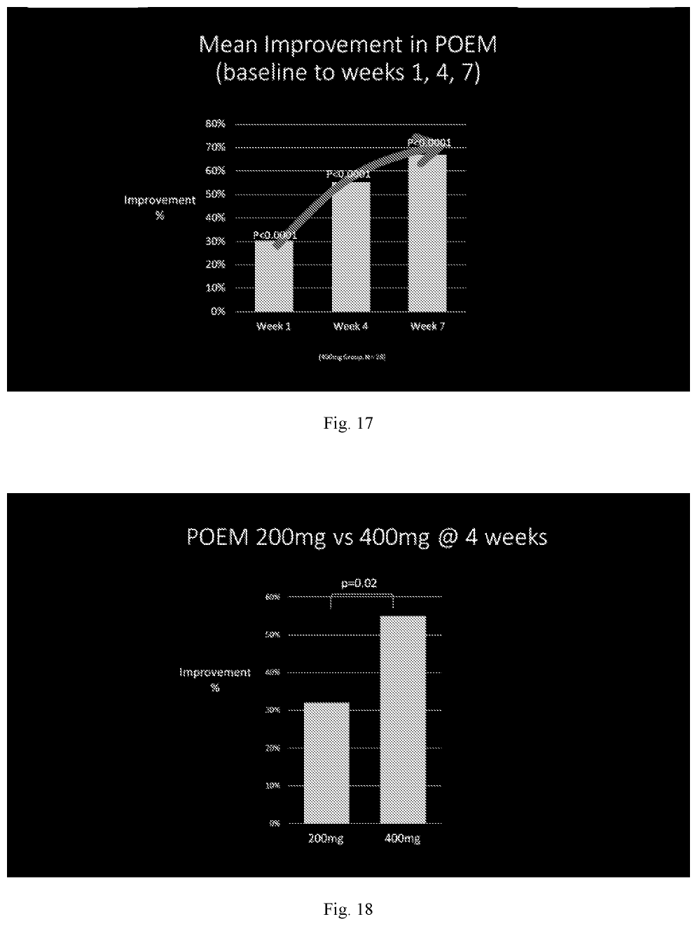

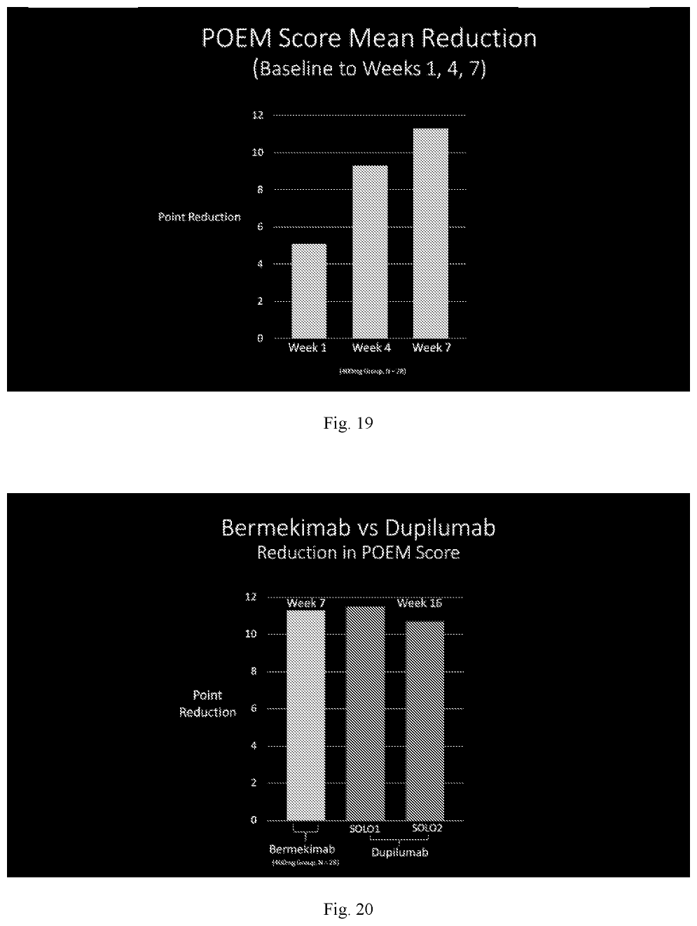

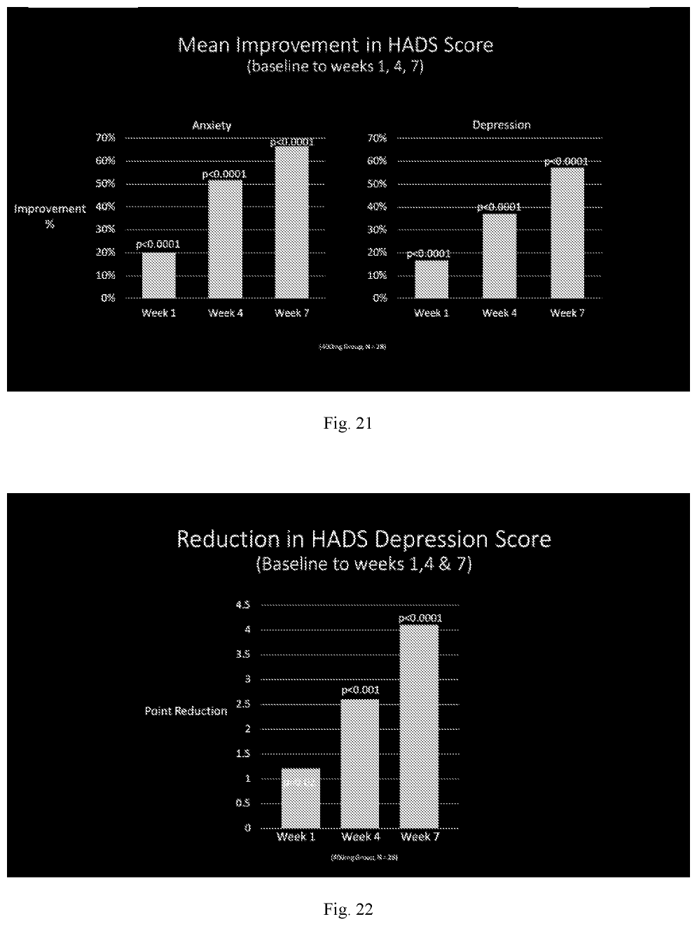

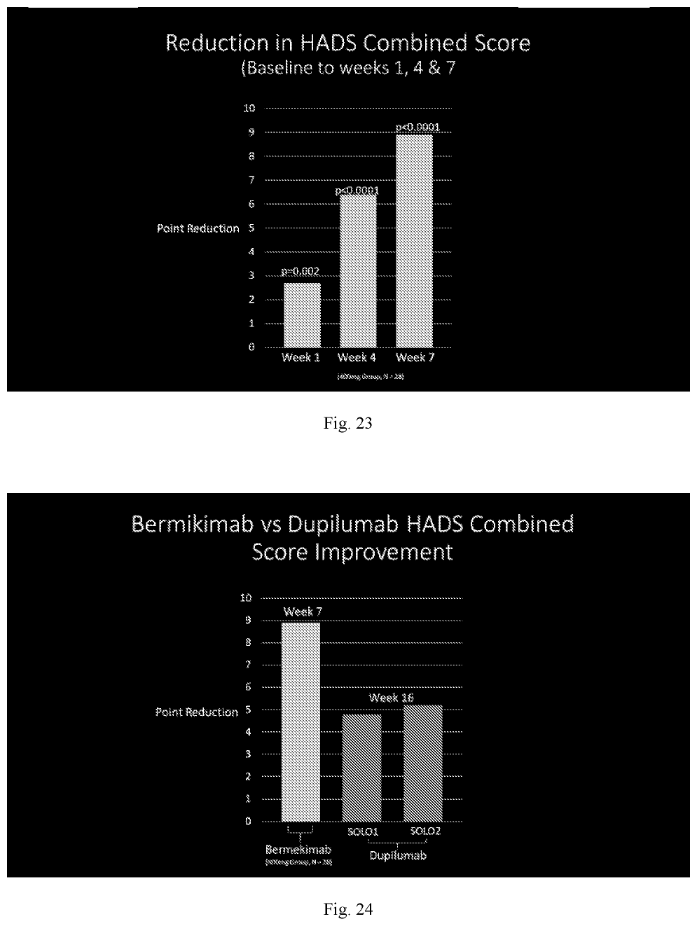

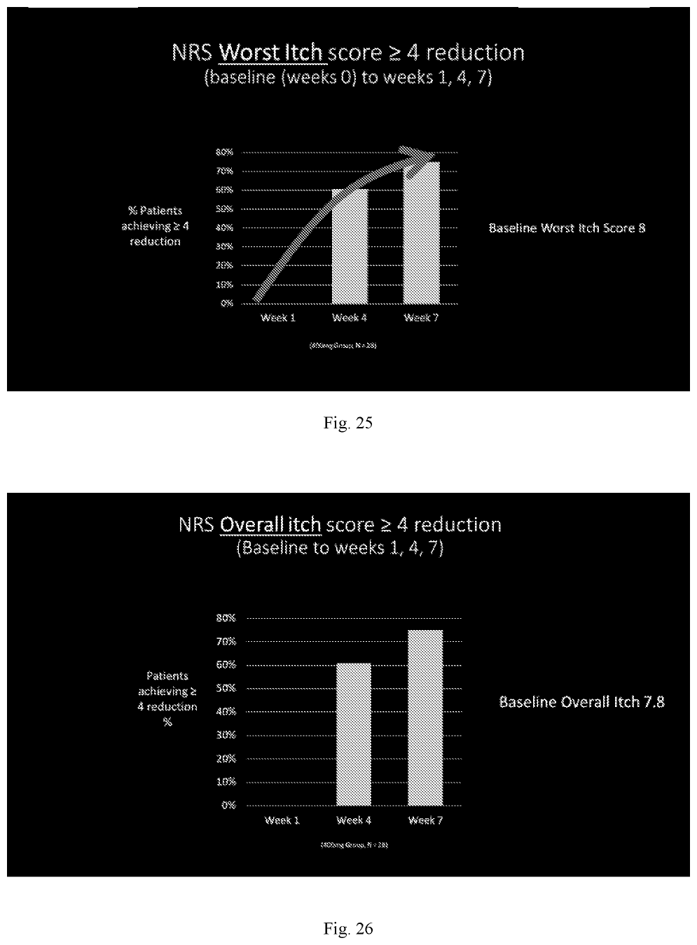

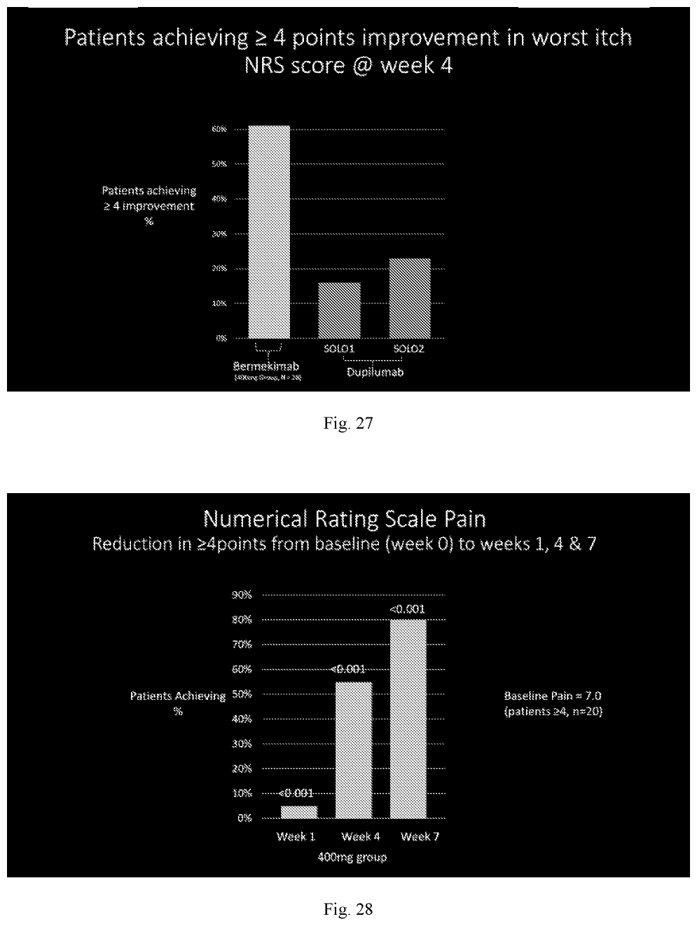

Phase 2, open label, dose escalation study of two dose cohorts of bermekimab in patients with moderate to severe atopic dermatitis. The study is multicenter, and consists of two dose levels: bermekimab administered subcutaneously at a dose of 200 mg weekly (4 doses) and bermekimab administered subcutaneously at a dose of 400 mg weekly (8 doses). Patients taking the 200 mg dose are followed for 5 weeks (6 visits, day 35+/−2), and patients taking the 400 mg dose are followed for 8 weeks (9 visits, day 56+/−2) to allow for assessment of safety and efficacy. The study calendar is shown in FIG. 4 where:
^{a} Chemistry Panel including: Albumin, Alkaline Phosphatase, ALT, AST, GGT, Bicarbonate (CO2) Calcium, Chloride, Creatinine, Glucose, Potassium, Sodium, Total Bilirubin, Total Protein, Urea Nitrogen.
^{b} Hematology Panel including: Complete whole blood (WBC, HgB, Platelet, differential).
^{c} Blood draw for PK and Biomarker analysis.
^{d} Data from patient diary for the previous 7 days to be recorded at this time.
^{e} Interferon gamma release assay.
^{f} BMI will be calculated at this visit using height and weight.
[symbol] HIV antibody, Hepatitis C antibody, Hepatitis B panel (HBsAg, anti-HBc, anti-HBs), and interferon gamma release assay (IGRA).
▴ Urinalysis will assess pH, protein, glucose, and blood cells.
£ A standard 12-lead ECG will be performed. The ECG strips and/or reports will be retained with the source documentation.
[symbol] Each bermekimab injection will be followed by 1 hour monitoring for injection site reaction and vital signs 1 hour post injection (70+/−10 minutes).
⁺ Vital signs include blood pressure, pulse, oxygen saturation, respiratory rate and body temperature.
[symbol] Assessment of patients Pruritus, Pain and Erythema will be recorded twice [once pre-injection, once post-injection of bermekimab] during visit 1.
* Concomitant medications within 30 days before screening until 7 days after the last administration of the study drug must be recorded for the purpose of drug-drug and drug-disease interaction evaluation and signal detection.

Study Endpoints

Primary Endpoint: Safety and Tolerability.

Secondary Endpoints:
Change in Eczema Area and Severity Index Score (EASI) from baseline to visit 8.
EASI score was used to assess severity and extent of AD with respect to erythema, excoriation, infiltration and lichenification at 4 anatomic sites of the body: lower and upper extremities, trunk and head. The total EASI score ranges from 0 to 72 points (from no disease to maximum disease severity, respectively).
Patients (%) achieving Investigator's Global Assessment (IGA) Response (0 or 1) at Visit 8. IGA assesses disease severity and clinical response using a 5-point scale: 0=clear; 1=almost clear; 2=mild; 3=moderate; 4=severe. The score is determined by ranking the extent of erythema and papulation/infiltration. A clinical response to therapy will be an IGA score of 0 (clear) or 1 (almost clear). Patients receiving more than one treatment with additional medication to treat AD exacerbation during the study or missing IGA scores at Visit 8 are treated as non-responders.
Patients (%) achieving ≥2 IGA Score Reduction at Visit 8.
Pharmacokinetics (PK) Assessment. An enzyme-linked immunosorbent assay (ELISA) has been developed to specifically measure bermekimab levels in human plasma.
Change (%) for peak weekly averaged pruritus numerical rating scores (NRS) from baseline to visit 8. The NRS rating system captures the intensity of patient's itch and pain over a 24-hour period. The following question was presented to patients: “how would a participant rate his or her itch at the worst moment and on average during the previous 24 hours (scale 0-10 [0=no itch; 10=worst possible itch])?” and “how would you rate your pain on average during the previous 24 hours [0=no pain; 10=severe pain])?”
Change in weekly averaged peak NRS from baseline to visit 8.
Change in SCORing Atopic Dermatitis (SCORAD) score from baseline to visit 8. SCORAD was developed by the European Task Force on Atopic Dermatitis (Seventy scoring of atopic dermatitis: the SCORAD index) as a measure of disease severity in AD. It includes assessment of the eczema in addition to patient reported symptoms. Total score ranges from 0 to 103 (no disease to most severe disease, respectively).
Patients (%) achieving 50% or greater reduction in EASI Score from baseline to Visit 8
Patients (%) achieving 50% or greater reduction in SCORAD Score to Visit 8
Change (%) in Patient Oriented Eczema Measure (POEM) Scores from baseline to Visit 8. POEM is a 7-item patient reported quality of life outcome measure based on a questionnaire to determine disease symptoms, including bleeding, cracking, dryness, flaking, itching, sleep loss and weeping. The scoring range is from 0 to 28 (no disease to most severe disease, respectively).
Changes in Global Individual Signs Score (GISS) from baseline to visit 8. GISS assesses AD lesions for erythema, excoriations, lichenification and edema/papulation. Each component will be rated on a global basis (over the entire body surface rather than by region) using a 4-point scale (0=none, 1=mild, 2=moderate and 3=severe) according to the EASI grading severity. Total score will range from 0 to 12 (no disease to most severe disease, respectively).
Change from baseline to visit 8 in Dermatology Life Quality Index (DLQI)
Change from baseline to visit 8 in Hospital Anxiety Depression Scale (HADS)
Change (%) from pre- and post-injection of Visit 1 Questionnaire for pruritus, pain and erythema

Bermekimab Therapy Rapidly and Significantly Reduces Disease.

Thirty-eight patients in two treatment groups received a low (n=10) or high (n=28) dose of bermekimab once weekly for either a 4 or 7-week treatment regimen, respectively. Statistically significant improvement was seen for all efficacy endpoints in the high dose group; and a significant dose response for the high dose compared to low dose group was observed for key endpoints, including the Eczema Area and Severity Index (EASI), Global Individual Sign Score (GISS), Patient Oriented Eczema Measure (POEM), Hospital Anxiety and Depression Scale (HADS), and SCORing Atopic Dermatitis (SCORAD).

While clinically and statistically significant improvement was seen for all clinical endpoints in the high dose group, also notable was the speed, magnitude, and trajectory of responses seen. In the high dose group, for example, after only four weeks of treatment, 61% of patients achieved a 4-point improvement in the Pruritus Numerical Rating Scale (NRS), a key method used to measure itch in clinical trials for atopic dermatitis, and 75% of patients achieved a 4-point improvement by week 7. For the only biological therapy currently approved to treat atopic dermatitis, dupilumab, which was granted breakthrough designation by the FDA, only 16%-23% of patients achieved a 4-point NRS improvement after 4 weeks of therapy; and only 36-41% of patients achieved a 4-point improvement by week 16. Simpson E L, Bieber T, Guttman-Yassky E, et al; SOLO 1 and SOLO 2 Investigators. Two phase 3 trials of dupilumab versus placebo in atopic dermatitis. N Engl J Med. 2016; 375(24):2335-2348.

Atopic dermatitis, commonly referred to as eczema, is characterized by chronic inflammation of the skin, which results in a breakdown of the skin barrier and leads to dry, thickened, scaly skin, redness, and itching, the latter which can be debilitating and result in significant sleep disturbances and loss of quality of life. A survey of persons suffering from atopic dermatitis found that 91% of patients endured itching every day (Dawn et al. Itch characteristics in atopic dermatitis: results of a web-based questionnaire. Br J Dermatol. 2009; 160(3):642-644), and another study reported that 36% of patients feel that their primary treatment objective is to reduce itch (Schmitt et al. Determinants of treatment goals and satisfaction of patients with atopic eczema. J Dtsch Dermatol Ges. 2008; 6(6):458-465). Further, international panels of dermatology experts have recommended itch as a crucial determinate of treatment effectiveness in the development of new therapies. Simpson et al. When does atopic dermatitis warrant systemic therapy? Recommendations from an expert panel of the International Eczema Council. J Am Acad Dermatol. 2017 October; 77(4):623-633.

Another key measure of efficacy in the study was the EASI. In the study, 39% of high dose patients achieved 75% improvement in EASI score (EASI-75) after 4 weeks of therapy and 71% of patients achieved EASI-75 at week 7. Of note, participants were not allowed to use concomitant topical corticosteroids during the study and thus these improvements were most likely due to the study drug alone. The only approved biological therapy, dupilumab, reports only 44-51% of patients achieved EASI-75 by week 16.

Dr. Eric Simpson, Professor of Dermatology at Oregon Health & Science University, commented on the bermekimab findings: “These early results with bermekimab are extremely exciting. Patients with moderate-to-severe atopic dermatitis achieved very clinically relevant improvement in not only skin signs, but multiple domains of their life impacted by this chronic disease. It is greatly encouraging to see that blocking the novel target, IL-1 alpha, yields such potent anti-inflammatory effects and treats the key aspects of this disease.” These views were shared by Dr. Alice Gottlieb, M.D., Ph.D., Professor of Dermatology at New York Medical College, who stated, “Bermekimab is a very promising new drug and I look forward to its continued development.” Dr. Seth Forman, an investigator in the clinical study in Tallahassee, Fla. stated, “Bermekimab provided relief to my patients with skin disease with excellent safety. I look forward to having bermekimab available for my atopic dermatitis patients in the future.”

This study evaluated a number of accepted measures of disease severity for atopic dermatitis, including the Eczema Area and Severity Index score (EASI); Dermatology Life Quality Index (DLQI); SCORAD; Pruritus Numerical Rating Scale (NRS); Patient Oriented Eczema Measure (POEM); The Hospital Anxiety and Depression Scale (HADS); and Investigator's Global Assessment (IGA). The two dose groups received weekly subcutaneous injections using XBiotech's recently developed pre-filled syringes that contain a concentrated formula of bermekimab. Improvement was assessed from baseline to the endpoint, which was either 4 or 7 weeks from start of treatment. Significant improvements were indicated by all aforementioned measures for the high dose group.

### Example 2—Formulation of an Anti-IL-1α mAb with Improved Bioavailability

The PK data analysis from the study described in Example 1 provided remarkable evidence of improved bioavailability in the 400 mg dosing (200 mg/ml formulation) cohort. Compare Tables 3 and 4 below.

TABLE 3
PK Results for 200 mg Dose Group

| | Bermekimab Plasma Concentration (μg/mL) | | | | |
|---|---|---|---|---|---|
| | Visit 1 pre | Visit 2 pre | Visit 3 pre | Visit 4 Pre | Visit 5 |
| | (day 0) | (day 7) | (day 14) | (day 21) | (day 28) |
| N | 9 | 8 | 8 | 8 | 6 |
| Mean | <0.1 | 6.1 | 10.6 | 11.1 | 12.6 |
| Median | <0.1 | 6.2 | 8.6 | 11.7 | 13.3 |

TABLE 4
PK Results for 400 mg Dose Group

Bermekimab Plasma Concentration (μg/mL)

| | Visit 1 pre | Visit 3 pre | Visit 5 Pre | Visit 8 |
|---|---|---|---|---|
| | (day 0) | (day 14) | (day 28) | (day 49) |
| N | 28 | 25 | 20 | 22 |
| Mean | <0.1 | 41.2 | 43.8 | 47.1 |
| Median | <0.1 | 37.8 | 40.6 | 40.9 |

The observed bermekimab plasma concentration from 400 mg dose group was observed to be 3-4 fold higher than that from 200 mg dose group at visit 3 pre dose and visit 5 pre dose as shown in Table 5 below.

TABLE 5
PK Results Comparison for Two Dose Groups

| | Bermekimab | Bermekimab | Bermekimab |
|---|---|---|---|
| | Plasma | Plasma | Plasma |
| | Concentration | Concentration | concentration |
| | (200 mg | (400 mg | fold |
| | Group) | Group) | increase |
| Visit 3 pre Mean | 10.6 | 41.2 | 3.9 |
| Visit 3 pre Median | 8.6 | 37.8 | 4.4 |
| Visit 5 pre Mean | 12.6 | 43.8 | 3.5 |
| Visit 5 pre Median | 13.3 | 40.6 | 3.1 |

Subjects in the study who received a 200 mg weekly dose (using a 100 mg/ml formulation) exhibited maximum measured (n=6) mean plasma levels of 13 ug/ml. The mean measured maximum plasma level (n=22) for patients who received a 400 mg/ml weekly dose (using a 200 mg/ml formulation) was 47 ug/ml. These findings show that when the weekly dose was doubled from 200 mg to 400 mg, the actual maximum plasma levels increased by 3.6 fold—which was a surprising and significant improvement in bioavailability.

The exposure-response correlation observed in these findings was most remarkable. The improvement in all four clinical endpoints exhibited linear relationship in correspondence with bermekimab plasma concentration. In other words, it is evident that dose dependent improvements are achieved in all four clinical outcomes. See Tables 6 and 7 below.

TABLE 6
Exposure-Response Results Compared with reduction at Week 4

| | | | | Comparing 400 | |
|---|---|---|---|---|---|
| | | Results at | Results at | mg vs. 200 mg | |
| | | Week 4 of | Week 4 of | Dose Group | |
| | Base- | 200 mg | 400 mg | Fold | P value |
| Dose (mg) | line | Dose Group | Dose Group | Change | (T-test) |
| Plasma | 0.1 | 12.6 | 43.8 | 3.5 | 0.003 |
| Concentration | | | | | |
| (Mean) | | | | | |
| SCORAD | 0 | 11 | 38.6 | 3.5 | 0.0002 |
| (Reduction | | | | | |
| at Week 4) | | | | | |
| EASI | 0 | 7.1 | 20 | 2.8 | 0.01 |
| (Reduction | | | | | |
| at Week 4) | | | | | |
| GISS | 0 | 0.9 | 4.6 | 5.1 | <0.0001 |
| (Reduction | | | | | |
| at Week 4) | | | | | |
| IGA | 0 | 0.5 | 1.2 | 2.4 | 0.02 |
| (Reduction | | | | | |
| at Week 4) | | | | | |

TABLE 7
Exposure-Response Results Compared
with % improvement at Week 4

| | | Results at | Results of | Results Fold |
|---|---|---|---|---|
| | | Week 4 of | Week 4 of | Change 400 mg |
| | Base- | 200 mg | 400 mg | vs. 200 mg |
| Dose (mg) | line | Dose Group | Dose Group | Dose Group |
| Plasma | 0.1 | 12.6 | 43.8 | 3.5 |
| Concentration | | | | |
| (Mean) | | | | |
| SCORAD | 0 | 19 | 54 | 2.8 |
| (% Improvement) | | | | |
| EASI | 0 | 25 | 66 | 2.6 |
| (% Improvement) | | | | |
| GISS | 0 | 11 | 47 | 4.3 |
| (% Improvement) | | | | |
| IGA | 0 | 14 | 36 | 2.6 |
| (% Improvement) | | | | |

The expectation of a simple 1:1 correlation for a dose effect on clinical outcomes, reflecting a doubling of the dose, was not observed. Rather, for all the efficacy measures used to assess disease severity—EASI, SCORAD, GISS and IGA—the improvement in disease reduction was markedly greater than what was expected for dose effect. The mean reduction in disease severity at week 4 (the latest time point used for the 200 mg cohort) across all the disease measures averaged 3.5 fold greater for the 400 mg vs 200 mg groups.

The estimated bioavailability is 61% for 200 mg dose group, but 94% for 400 mg dose group. The 400 mg dose group used a newly developed formulation of 200 mg/mL, while the 200 mg dose group used the formulation of 100 mg/mL. This new formulation of 200 mg/mLis observed to have higher viscosity (38.2 cP measured at 25° C.). The higher viscosity, though, may help with the resistance to fluid flow through the interstitium who already has high viscosity due to tight association of water to hyaluronic acid. On the other hand, the lymphatic capillaries are blind-ended and composed of a single layer of overlapping endothelial cells, and lack tight cell-cell junctions as well as a continuous basement membrane. Increase in interstitial pressure stretches the fibers and leads to an opening of lymphatic lumen, which allows easy entry of large-molecular-weight solutes. The increase in viscosity and drug concentration in the new formulation may result in the increase of interstitial pressure and make the new formulation easier to be absorbed into a lymphatic system. The faster absorption of 400 mg dose group is confirmed with the observation that a steady state of plasma concentration is achieved after only two treatment cycles, as the accumulation starting from cycle three is not more than 4% for each cycle. On the other hand, in the 200 mg dose group, the steady state is barely achieved at the end of the study (the fourth treatment cycle).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1-10. (canceled)

11. A method of reducing a symptom of atopic dermatitis in a human subject with atopic dermatitis, the method comprising the step of administering to the subject a pharmaceutical composition comprising a pharmaceutically acceptable carrier and a therapeutically effective amount of an agent that selectively binds IL-1α at least until the symptom of atopic dermatitis in the subject is reduced.

12. The method of claim 11, wherein the agent is an anti-IL-1α antibody.

13. The method of claim 12, wherein the anti-IL-1α antibody is a monoclonal antibody.

14. The method of claim 13, wherein the monoclonal antibody is an IgG1.

15. The method of claim 13, wherein the monoclonal antibody comprises a complementarity determining region of bermekimab.

16. The method of claim 13, wherein the monoclonal antibody is bermekimab.

17. The method of claim 11, wherein the symptom of atopic dermatitis is pruritis.

18. The method of claim 11, wherein the symptom of atopic dermatitis is pain.

19-20. (canceled)
